SEQUENCE LISTING (17 sequences)

SEQ ID NO: 1 - DNA, 633 bp, Mus musculus
  source 1..633: mol_type = other DNA
atgaagttcctctctgcaagagacttccatccagttgccttcttgggactgatgctggtgacaaccacggccttccctacttcacaagtccggagaggagacttcacagaggataccactcccaacagacctgtctataccacttcacaagtcggaggcttaattacacatgttctctgggaaatcgtggaaatgagaaaagagttgtgcaatggcaattctgattgtatgaacaacgatgatgcacttgcagaaaacaatctgaaacttccagagatacaaagaaatgatggatgctaccaaactggatataatcaggaaatttgcctattgaaaatttcctctggtcttctggagtaccatagctacctggagtacatgaagaacaacttaaaagataacaagaaagacaaagccagagtccttcagagagatacagaaactctaattcatatcttcaaccaagaggtaaaagatttacataaaatagtccttcctaccccaatttccaatgctctcctaacagataagctggagtcacagaaggagtggctaaggaccaagaccatccaattcatcttgaaatcacttgaagaatttctaaaagtcactttgagatctactcggcaaacc

SEQ ID NO: 2 - protein, 211 aa, Mus musculus
MKFLSARDFHPVAFLGLMLVTTTAFPTSQVRRGDFTEDTTPNRPVYTTSQVGGLITHVLWEIVEMRKELCNGNSDCMNNDDALAENNLKLPEIQRNDGCYQTGYNQEICLLKISSGLLEYHSYLEYMKNNLKDNKKDKARVLQRDTETLIHIFNQEVKDLHKIVLPTPISNALLTDKLESQKEWLRTKTIQFILKSLEEFLKVTLRSTRQT

SEQ ID NO: 3 - DNA, 459 bp, Mus musculus
  source 1..459: mol_type = other DNA
gttcccattagacaactgcactacaggctccgagatgaacaacaaaaaagcctcgtgctgtcggacccatatgagctgaaagctctccacctcaatggacagaatatcaaccaacaagtgatattctccatgagctttgtacaaggagaaccaagcaacgacaaaatacctgtggccttgggcctcaaaggaaagaatctatacctgtcctgtgtaatgaaagacggcacacccaccctgcagctggagagtgtggatcccaagcaatacccaaagaagaagatggaaaaacggtttgtcttcaacaagatagaagtcaagagcaaagtggagtttgagtctgcagagttccccaactggtacatcagcacctcacaagcagagcacaagcctgtcttcctgggaaacaacagtggtcaggacataattgacttcaccatggaatccgtgtcttcctaa

SEQ ID NO: 4 - protein, 152 aa, Mus musculus
VPIRQLHYRLRDEQQKSLVLSDPYELKALHLNGQNINQQVIFSMSFVQGEPSNDKIPVALGLKGKNLYLSCVMKDGTPTLQLESVDPKQYPKKKMEKRFVFNKIEVKSKVEFESAEFPNWYISTSQAEHKPVFLGNNSGQDIIDFTMESVSS

SEQ ID NO: 5 - DNA, 48 bp, synthetic construct
  source 1..48: mol_type = other DNA
tctggtggcggaggctcgggcggaggtgggtcgggtggcggcggatca

SEQ ID NO: 6 - protein, 16 aa, synthetic construct
SGGGGSGGGGSGGGGS

SEQ ID NO: 7 - DNA, 1140 bp, synthetic construct
  source 1..1140: mol_type = other DNA
atgaagttcctctctgcaagagacttccatccagttgccttcttgggactgatgctggtgacaaccacggccttccctacttcacaagtccggagaggagacttcacagaggataccactcccaacagacctgtctataccacttcacaagtcggaggcttaattacacatgttctctgggaaatcgtggaaatgagaaaagagttgtgcaatggcaattctgattgtatgaacaacgatgatgcacttgcagaaaacaatctgaaacttccagagatacaaagaaatgatggatgctaccaaactggatataatcaggaaatttgcctattgaaaatttcctctggtcttctggagtaccatagctacctggagtacatgaagaacaacttaaaagataacaagaaagacaaagccagagtccttcagagagatacagaaactctaattcatatcttcaaccaagaggtaaaagatttacataaaatagtccttcctaccccaatttccaatgctctcctaacagataagctggagtcacagaaggagtggctaaggaccaagaccatccaattcatcttgaaatcacttgaagaatttctaaaagtcactttgagatctactcggcaaacctctggtggcggaggctcgggcggaggtgggtcgggtggcggcggatcagttcccattagacaactgcactacaggctccgagatgaacaacaaaaaagcctcgtgctgtcggacccatatgagctgaaagctctccacctcaatggacagaatatcaaccaacaagtgatattctccatgagctttgtacaaggagaaccaagcaacgacaaaatacctgtggccttgggcctcaaaggaaagaatctatacctgtcctgtgtaatgaaagacggcacacccaccctgcagctggagagtgtggatcccaagcaatacccaaagaagaagatggaaaaacggtttgtcttcaacaagatagaagtcaagagcaaagtggagtttgagtctgcagagttccccaactggtacatcagcacctcacaagcagagcacaagcctgtcttcctgggaaacaacagtggtcaggacataattgacttcaccatggaatccgtgtcttcctaa

SEQ ID NO: 8 - protein, 379 aa, synthetic construct
MKFLSARDFHPVAFLGLMLVTTTAFPTSQVRRGDFTEDTTPNRPVYTTSQVGGLITHVLWEIVEMRKELCNGNSDCMNNDDALAENNLKLPEIQRNDGCYQTGYNQEICLLKISSGLLEYHSYLEYMKNNLKDNKKDKARVLQRDTETLIHIFNQEVKDLHKIVLPTPISNALLTDKLESQKEWLRTKTIQFILKSLEEFLKVTLRSTRQTSGGGGSGGGGSGGGGSVPIRQLHYRLRDEQQKSLVLSDPYELKALHLNGQNINQQVIFSMSFVQGEPSNDKIPVALGLKGKNLYLSCVMKDGTPTLQLESVDPKQYPKKKMEKRFVFNKIEVKSKVEFESAEFPNWYISTSQAEHKPVFLGNNSGQDIIDFTMESVSS

SEQ ID NO: 9 - DNA, 636 bp, Homo sapiens
  source 1..636: mol_type = other DNA
atgaactccttctccacaagcgccttcggtccagttgccttctccctggggctgctcctggtgttgcctgctgccttccctgccccagtacccccaggagaagattccaaagatgtagccgccccacacagacagccactcacctcttcagaacgaattgacaaacaaattcggtacatcctcgacggcatctcagccctgagaaaggagacatgtaacaagagtaacatgtgtgaaagcagcaaagaggcactggcagaaaacaacctgaaccttccaaagatggctgaaaaagatggatgcttccaatctggattcaatgaggagacttgcctggtgaaaatcatcactggtcttttggagtttgaggtatacctagagtacctccagaacagatttgagagtagtgaggaacaagccagagctgtgcagatgagtacaaaagtcctgatccagttcctgcagaaaaaggcaaagaatctagatgcaataaccacccctgacccaaccacaaatgccagcctgctgacgaagctgcaggcacagaaccagtggctgcaggacatgacaactcatctcattctgcgcagctttaaggagttcctgcagtccagcctgagggctcttcggcaaatg

SEQ ID NO: 10 - protein, 212 aa, Homo sapiens
MNSFSTSAFGPVAFSLGLLLVLPAAFPAPVPPGEDSKDVAAPHRQPLTSSERIDKQIRYILDGISALRKETCNKSNMCESSKEALAENNLNLPKMAEKDGCFQSGFNEETCLVKIITGLLEFEVYLEYLQNRFESSEEQARAVQMSTKVLIQFLQKKAKNLDAITTPDPTTNASLLTKLQAQNQWLQDMTTHLILRSFKEFLQSSLRALRQM

SEQ ID NO: 11 - DNA, 462 bp, Homo sapiens
  source 1..462: mol_type = other DNA
gcacctgtacgatcactgaactgcacgctccgggactcacagcaaaaaagcttggtgatgtctggtccatatgaactgaaagctctccacctccagggacaggatatggagcaacaagtggtgttctccatgtcctttgtacaaggagaagaaagtaatgacaaaatacctgtggccttgggcctcaaggaaaagaatctgtacctgtcctgcgtgttgaaagatgataagcccactctacagctggagagtgtagatcccaaaaattacccaaagaagaagatggaaaagcgatttgtcttcaacaagatagaaatcaataacaagctggaatttgagtctgcccagttccccaactggtacatcagcacctctcaagcagaaaacatgcccgtcttcctgggagggaccaaaggcggccaggatataactgacttcaccatgcaatttgtgtcttcctaa

SEQ ID NO: 12 - protein, 153 aa, Homo sapiens
APVRSLNCTLRDSQQKSLVMSGPYELKALHLQGQDMEQQVVFSMSFVQGEESNDKIPVALGLKEKNLYLSCVLKDDKPTLQLESVDPKNYPKKKMEKRFVFNKIEINNKLEFESAQFPNWYISTSQAENMPVFLGGTKGGQDITDFTMQFVSS

SEQ ID NO: 13 - DNA, 1146 bp, synthetic construct
  source 1..1146: mol_type = other DNA
atgaactccttctccacaagcgccttcggtccagttgccttctccctggggctgctcctggtgttgcctgctgccttccctgccccagtacccccaggagaagattccaaagatgtagccgccccacacagacagccactcacctcttcagaacgaattgacaaacaaattcggtacatcctcgacggcatctcagccctgagaaaggagacatgtaacaagagtaacatgtgtgaaagcagcaaagaggcactggcagaaaacaacctgaaccttccaaagatggctgaaaaagatggatgcttccaatctggattcaatgaggagacttgcctggtgaaaatcatcactggtcttttggagtttgaggtatacctagagtacctccagaacagatttgagagtagtgaggaacaagccagagctgtgcagatgagtacaaaagtcctgatccagttcctgcagaaaaaggcaaagaatctagatgcaataaccacccctgacccaaccacaaatgccagcctgctgacgaagctgcaggcacagaaccagtggctgcaggacatgacaactcatctcattctgcgcagctttaaggagttcctgcagtccagcctgagggctcttcggcaaatgtctggtggcggaggctcgggcggaggtgggtcgggtggcggcggatcagcacctgtacgatcactgaactgcacgctccgggactcacagcaaaaaagcttggtgatgtctggtccatatgaactgaaagctctccacctccagggacaggatatggagcaacaagtggtgttctccatgtcctttgtacaaggagaagaaagtaatgacaaaatacctgtggccttgggcctcaaggaaaagaatctgtacctgtcctgcgtgttgaaagatgataagcccactctacagctggagagtgtagatcccaaaaattacccaaagaagaagatggaaaagcgatttgtcttcaacaagatagaaatcaataacaagctggaatttgagtctgcccagttccccaactggtacatcagcacctctcaagcagaaaacatgcccgtcttcctgggagggaccaaaggcggccaggatataactgacttcaccatgcaatttgtgtcttcctaa

SEQ ID NO: 14 - protein, 381 aa, synthetic construct
MNSFSTSAFGPVAFSLGLLLVLPAAFPAPVPPGEDSKDVAAPHRQPLTSSERIDKQIRYILDGISALRKETCNKSNMCESSKEALAENNLNLPKMAEKDGCFQSGFNEETCLVKIITGLLEFEVYLEYLQNRFESSEEQARAVQMSTKVLIQFLQKKAKNLDAITTPDPTTNASLLTKLQAQNQWLQDMTTHLILRSFKEFLQSSLRALRQMSGGGGSGGGGSGGGGSAPVRSLNCTLRDSQQKSLVMSGPYELKALHLQGQDMEQQVVFSMSFVQGEESNDKIPVALGLKEKNLYLSCVLKDDKPTLQLESVDPKNYPKKKMEKRFVFNKIEINNKLEFESAQFPNWYISTSQAENMPVFLGGTKGGQDITDFTMQFVSS

SEQ ID NO: 15 - DNA, 9635 bp, synthetic construct
  source 1..9635: mol_type = other DNA
aatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgctagcttttaaaagaaaaggggggattggggggtacagtgcaggggaaagaatagtagacataatagcaacagacatacaaactaaagaattacaaaaacaaattacaaaaattcaaaattttactagtgattatcggatcaactttgtatagaaaagttgggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggtctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgacaagtttgtacaaaaaagcaggctgccaccatgaagttcctctctgcaagagacttccatccagttgccttcttgggactgatgctggtgacaaccacggccttccctacttcacaagtccggagaggagacttcacagaggataccactcccaacagacctgtctataccacttcacaagtcggaggcttaattacacatgttctctgggaaatcgtggaaatgagaaaagagttgtgcaatggcaattctgattgtatgaacaacgatgatgcacttgcagaaaacaatctgaaacttccagagatacaaagaaatgatggatgctaccaaactggatataatcaggaaatttgcctattgaaaatttcctctggtcttctggagtaccatagctacctggagtacatgaagaacaacttaaaagataacaagaaagacaaagccagagtccttcagagagatacagaaactctaattcatatcttcaaccaagaggtaaaagatttacataaaatagtccttcctaccccaatttccaatgctctcctaacagataagctggagtcacagaaggagtggctaaggaccaagaccatccaattcatcttgaaatcacttgaagaatttctaaaagtcactttgagatctactcggcaaacctctggtggcggaggctcgggcggaggtgggtcgggtggcggcggatcagttcccattagacaactgcactacaggctccgagatgaacaacaaaaaagcctcgtgctgtcggacccatatgagctgaaagctctccacctcaatggacagaatatcaaccaacaagtgatattctccatgagctttgtacaaggagaaccaagcaacgacaaaatacctgtggccttgggcctcaaaggaaagaatctatacctgtcctgtgtaatgaaagacggcacacccaccctgcagctggagagtgtggatcccaagcaatacccaaagaagaagatggaaaaacggtttgtcttcaacaagatagaagtcaagagcaaagtggagtttgagtctgcagagttccccaactggtacatcagcacctcacaagcagagcacaagcctgtcttcctgggaaacaacagtggtcaggacataattgacttcaccatggaatccgtgtcttcctaaacccagctttcttgtacaaagtggtgataatcgaattccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgggaattcccgcggttcgaattctaccgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgacctcacgtggccaccatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgaggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagagagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagctt

SEQ ID NO: 16 - protein, 5 aa, synthetic construct
GGGGS

SEQ ID NO: 17 - protein, 17 aa, synthetic construct
AEAAAKEAAAKEAAAKA

(19) United States
(12) Patent Application Publication
(10) Pub. No.: US 2024/0166706 A1
(43) Pub. Date: May 23, 2024

(54) SYNTHETIC IL6-IL1BETA FUSION CYTOKINE FOR PROMOTING T CELL CYTOTOXIC FUNCTION, T CELL PROLIFERATION, AND TUMORICIDAL ACTIVITY

(71) Applicant: Wisconsin Alumni Research Foundation, Madison, WI (US)

(72) Inventors: Jacques Galipeau, Madison, WI (US); Rahul Das, Madison, WI (US)

(21) Appl. No.: 18/516,561

(22) Filed: Nov. 21, 2023

Related U.S. Application Data
(60) Provisional application No. 63/426,962, filed on Nov. 21, 2022.
(60) Provisional application No. 63/508,769, filed on Jun. 16, 2023.

(51) Int. Cl. C07K 14/54; A61K 39/00; A61P 35/00; C07K 14/545; C12N 5/0783

(52) U.S. Cl. CPC C07K 14/5412; A61K 39/4611; A61K 39/4635; A61K 39/464499; A61P 35/00; C07K 14/545; C12N 5/0638; A61K 38/00; A61K 2239/26; A61K 2239/39; A61K 2239/59; C07K 2319/00; C12N 2510/00

(57) ABSTRACT

The present invention provides novel fusion proteins comprising two cytokines: interleukin-6 (IL-6) and interleukin-1 beta (IL-1β). Methods of using the fusion proteins to activate target cells or treat a disease are also provided.

### CROSS-REFERENCE TO RELATED APPLICATIONS

This application claims priority to U.S. Provisional Application No. 63/426,962 filed on Nov. 21, 2022, and U.S. Provisional Application No. 63/508,769 filed on Jun. 16, 2023, the contents of both of which are incorporated by reference in their entireties.

### SEQUENCE LISTING

This application includes a sequence listing in XML format titled “96029604447_ST26.xml”, which is 31,337 bytes in size and was created on Nov. 20, 2023. The sequence listing is electronically submitted with this application via Patent Center and is incorporated herein by reference in its entirety.

### BACKGROUND

Several pro-inflammatory cytokines exhibit potent tumoricidal activities in vitro. However, cytokine monotherapies have shown only modest benefits for the treatment of cancer. It may be that any single cytokine is unlikely to provide a sufficient immune stimulus to overcome the numerous immune evasion mechanisms deployed by tumors, including the exhaustion of T cells. Thus, cytokine-based therapies offer untapped potential for use in cancer treatments.

### SUMMARY

In a first aspect, the present invention provides fusion proteins comprising from N-terminus to C-terminus: (a) an interleukin-6 (IL-6) peptide; (b) a linker peptide; and (c) an interleukin-1 beta (IL-1β) peptide.

In a second aspect, the present invention provides polynucleotides encoding a fusion protein described herein.

In a third aspect, the present invention provides cells comprising a polynucleotide described herein. Under suitable conditions, the cells express a fusion protein described herein.

In a fourth aspect, the present invention provides methods of generating a cell that expresses a fusion protein described herein. The methods comprise introducing a polynucleotide that encodes the fusion protein into the cell.

In a fifth aspect, the present invention provides methods of activating a target cell. The methods comprise contacting the target cell with a fusion protein described herein.

In a sixth aspect, the present invention provides methods of treating a disease in a subject. The methods comprise administering a fusion protein, polynucleotide, or cell described herein to the subject.

### BRIEF DESCRIPTION OF THE DRAWINGS

FIG. 1 shows a vector map of a lentiviral expression vector encoding the IL-6/1 fusokine. A detailed description of the vector components is provided in Table 1.

FIG. 2 shows the DNA and amino acid sequences of the IL-6/1 fusokine tested in the Examples. From N-terminus to C-terminus, this fusion protein comprises: the full-length mouse IL-6 protein (SEQ ID NO: 2), a 16-amino acid flexible peptide linker (SEQ ID NO: 6), and a portion of the mouse IL-1β protein (SEQ ID NO: 4). The IL-6 component is shown in bold font and the IL-1β component is shown in underlined font.

FIG. 3 shows the predicted ribbon structure of the IL-6/1 fusokine. This structure was obtained using Rosetta Ab Initio structure prediction software.

FIG. 4 shows detection of IL-6/1 and IL-1/6 fusokine via western blot. HEK cells were stably transduced with constructs encoding IL-6/1 or IL-1/6. Media was collected from the cell culture after 24 or 48 hours of culturing, and the cells were lysed after 48 hours. The media and lysates were resolved on a 10% reducing-denaturing SDS-PAGE gel. Protein was transferred to a nitrocellulose membrane and probed with antibodies against IL-1β (left) and IL-6 (right).

FIG. 5 demonstrates that the IL-6/1 fusokine enhances Dynabead-mediated activation of human peripheral blood T cells. Human peripheral blood mononuclear cells (PBMC) were collected from a healthy donor and CD3⁺ T cells were purified. The cells were labeled with CellTrace™ Far Red (CTFR) dye to track proliferation. The cells were then treated with CD3-CD28 Dynabeads® (magnetic beads) alone or in combination with IL-2 cytokine for about 96 hours (i.e., to induce proliferation) in the presence of IL-6, IL-1β, a combination of IL-6 and IL-1β, or the IL-6/1 fusokine. Cell proliferation was assessed using flow cytometry. Boxes highlight cells with the highest fluorescence, which were deemed to be non-proliferating cells. Treatment with the IL-6/1 fusokine massively reduced this peak.

FIGS. 6A-6E demonstrates that the IL-6/1 fusokine differentially affects the proliferation of human CD4⁺ and CD8⁺ T cells. Human PBMCs were collected from three healthy donors and CD3⁺ T cells were purified. The cells were labeled with CTFR dye to track proliferation. The cells were then activated via treatment with an anti-CD3/anti-CD28 antibody cocktail alone or in combination with IL-2 cytokine (i.e., to induce proliferation) for about 96 hours in the presence of IL-6, IL-1β, a combination of IL-6 and IL-1β, or the IL-6/1 fusokine. Cells were stained with anti-CD4 and anti-CD8 antibodies and cell proliferation was assessed using flow cytometry. Cell division (i.e., generation number) was assessed using the FCS Express Flow Cytometry Software. (A) CD4⁺ cell proliferation profile. (B) Quantification of CD4⁺ cells in different generations. (C) Percent of CD4⁺ cells that remain undivided in each treatment group. (D) CD8⁺ cell proliferation profile. (E) Quantification of CD8⁺ cells in different generations.

FIGS. 7A-7B demonstrates that the IL-6/1 fusokine protects T cells from activation-induced apoptosis. Human PBMCs were collected from three healthy donors and CD3⁺ T cells were purified. Cells were activated via treatment with IL-6, IL-1β, a combination of IL-6 and IL-1β, or the IL-6/1 fusokine. After 96 hours of culturing, cells were stained with annexin and DAPI to determine the degree of apoptosis using flow cytometry. (A) Flow cytometry plots generated with cells from each of the three donors. The schematic diagram at the top shows the distribution of healthy, apoptotic, and necrotic cells in the flow cytometry plots. (B) Quantification of results shown in A. Asterisks indicate statistical significance (p<0.05) based on one-way analysis of variance (ANOVA) followed by Dunnet's test.

FIG. 8 demonstrates that the IL-6/1 fusokine selectively activates the STAT3-Akt signaling pathway in activated human PBMC T cells. Human PBMCs were collected from a healthy donor and CD3⁺ T cells were purified. Cells were induced to proliferate using anti-CD3 and anti-CD28 antibodies in the presence of IL-6, IL-1β, a combination of IL-6 and IL-1β, or the IL-6/1 fusokine. Conditioned media obtained from mock vector-transduced HEK cells (ctrl. media) was used as a control for the effects of proteins secreted by wild-type HEK cells. After 72 hours of culturing, cells were collected, lysed, and subjected to western blot to assess the effects of the treatments on activation of the JAK-STAT signaling pathway. Left: Western blot probed for phosphorylated-STAT3 (pSTAT3), total STAT3 (tSTAT3), phosphorylated-Akt (pAkt), total Akt (tAkt), and glyceraldehyde-3-phosphate dehydrogenase (GAPDH; loading control). Right: Western blot probed for phosphorylated-STAT1 (pSTAT1), phosphorylated-STAT5 (pSTAT5), p42/44 MAPK, and C-Jun.

FIG. 9 demonstrates that the IL-6/1 fusokine induces murine CD8⁺ tumor infiltrating lymphocyte (TIL) proliferation. A mouse model of ovarian cancer was generated by injecting 1D8 carcinoma cells into mice. TIL-containing ascetic fluid was collected from the mice (n=8) about 60 days after injection. TIL-T cells were purified and transduced with control lentivirus or lentivirus encoding the IL-6/1 fusokine. After 96 hours of culturing, cells were stained for CD4 and CD8 and analyzed by flow cytometry. While the number of CD4+ cells was about the same after control and fusokine treatment, the number of CD8+ cells increased.

FIG. 10 demonstrates that IL-6/1-transduced TIL-T cells have strong in vitro cytotoxic activity against murine ovarian cancer cells. A mouse model of ovarian cancer was generated by injecting ID8 carcinoma cells into mice. TIL-containing ascetic fluid was collected from the mice (n=10) about 60 days after injection. CD3⁺ TIL-T cells were purified and transduced with control lentivirus or lentivirus encoding the IL-6/1 fusokine. Transduced cells were co-cultured with 1D8 cells in a 1:10 ratio (ID8 cells: transduced cells). After 72 hours of culturing, cells were stained for CD45 (i.e., to distinguish between 1D8 and T cells) as well as annexin and 7AAD (i.e., to detect apoptosis). CD45⁻ cells (i.e., ID8 cells) were analyzed by flow cytometry.

FIG. 11 shows that IL-6/1-transduced TTL-T cells have tumoricidal activity against murine ovarian cancer ID8 cells in vivo. A mouse model of ovarian cancer was generated by injecting ID8 carcinoma cells into mice. TIL-containing ascetic fluid was collected from the mice (n=10) about 60 days after injection. CD3⁺ TIL-T cells were purified and transduced with lentivirus encoding the IL-6/1 fusokine. Then, another batch of mice were injected with 1×10⁶ luciferase-expressing 1D8 cells (Luc⁺-ID8). After 4 days, these mice (n=5/group) were injected with PBS (control group) or with TTL-T cells transduced with IL-6/1 (IL-6/1 group, 0.25×10⁶ cell/mouse). In vivo imaging was performed once every week. Left: Representative images showing luciferase signal in the mice. Right: Quantification of luciferase signal. Asterisk indicates statistical significance (p<0.05, Student's T test).

### DETAILED DESCRIPTION

The present invention provides a novel fusion protein comprising two cytokines: interleukin-6 (IL-6) and interleukin-1 beta (IL-1β). This fusion protein is referred to herein as “the IL-6/1 fusokine” or simply “IL-6/1”. IL-6 and IL-1β have complementary activities that support activation and survival of T cells. Within the IL-6/1 fusokine, these cytokines act synergistically to stimulate the immune system and may exhibit altered pharmacokinetics, biodistribution, and pharmacodynamics as compared to the cytokines individually.

In the Examples, the inventors demonstrate that IL-6/1 promotes T cell proliferation, protects T cells against apoptosis, and has tumoricidal effects both in vitro and in vivo. Specifically, they show that, when polynucleotides encoding IL-6/1 are transduced into tumor infiltrating lymphocytes (TILs) isolated from a mouse model of ovarian cancer, the TILs kill tumor cells when injected back into the mice.

IL-6/1 may be useful for several applications. For example, because IL-6/1 supports the growth and activity of T cells, it may be used to protect T cells grown in culture, as these cells are fragile ex vivo. Additionally, IL-6/1 may be expressed in cytotoxic lymphoid cells to make them resistant to the immunosuppressant tumor microenvironment. IL-6/1 may also be used as an adjuvant for T cell-based therapies, such as chimeric antigen receptor (CAR) T cell-based therapies. While CAR T cells produce satisfactory results when they are used to treat hematologic malignancies, they have been found to be ineffective against solid tumors. The problem may be that full activation of T cells requires not only the T cell receptor and costimulatory signals provided by the CAR T cells, but also the synergistic action of several cytokines. Thus, IL-6/1 could potentially be used to improve the efficacy of CAR T cells by providing dual cytokine signaling, allowing these cells to overcome the immunosuppressive microenvironment of solid tumors. Finally, IL-6/1 may be useful as a stand-alone therapeutic.

### Fusion Proteins:

In a first aspect, the present invention provides fusion proteins comprising from N-terminus to C-terminus: (a) an interleukin-6 (IL-6) peptide; (b) a linker peptide; and (c) an interleukin-1 beta (IL-1β) peptide.

As used herein, the term “fusion protein” refers to a single polypeptide comprising at least two peptide components (e.g., an IL-6 component and an IL-1β component). Each peptide component may be synthetic or naturally occurring. Naturally occurring components may comprise a full-length protein or a fragment thereof and may comprise mutations or other modifications relative to the wild-type protein from which they are derived. The fusion proteins of the present invention are referred to as “fusokines” because they comprise two peptide components that are derived from cytokines.

Interleukin-6 (IL-6) is a pleiotropic proinflammatory cytokine that is secreted by monocytes in response to infections and tissue injuries. IL-6 affects T cell activation, amplification, survival, and polarization¹⁻³. It promotes the expression of T cell attractor chemokines, prevents apoptosis in T cells via activation of STAT3 signaling, and plays a critical role in the accumulation of myeloid derived suppressor cells in tumor microenvironments.

The IL-6 receptor is a cell surface type I cytokine receptor complex that consists of the IL-6Rα chain (also known as CD126) and the signal-transducing component gp130 (also known as CD130). gp130 is a common signal transducer for several cytokines that is expressed in most tissues. In contrast, IL-6Rα chain expression is restricted to certain tissues, which include T-cells, monocytes, activated B-cells, and neutrophils. When IL-6 interacts with its receptor, it causes gp130 and IL-6R proteins to form a complex, which activates the receptor and initiates a signal transduction cascade.

The IL-6 peptide used in the fusion protein of the present invention need not be the full-length IL-6 protein. However, the IL-6 peptide should include, at a minimum, the IL-6 receptor binding domain, and it should retain the ability to bind to and activate the IL-6 receptor. The ability of an IL-6 peptide to bind to the IL-6 receptor may be assessed using any protein-protein binding assay, including those that utilize surface plasmon resonance, co-immunoprecipitation, or fluorescence resonance energy transfer (FRET). Alternatively, the ability of an IL-6 peptide to bind to the IL-6 receptor may be assessed using in silico modeling.

The IL-6 peptide may be derived from an IL-6 protein from any vertebrate animal. Suitable sources of IL-6 proteins include, but are not limited to, humans, non-human primates, cows, cats, dogs, pigs, and rodents. The inventors used the full-length mouse IL-6 protein (SEQ ID NO: 2) in the fusion protein tested in the Examples. Thus, in some embodiments, the IL-6 peptide has at least 90%, 92%, 94%, 95%, 96%, 98%, or 99% identity to SEQ ID NO:2. In other embodiments, the IL-6 peptide has at least 90%, 92%, 94%, 95%, 96%, 98%, or 99% identity to the human IL-6 protein (SEQ ID NO:10).

Interleukin-1 beta (IL-1β) is a potent pro-inflammatory cytokine that is crucial for host-defense responses to infection and injury. It is produced and secreted by a variety of cell types, including monocytes and macrophages. IL-1β promotes (1) expansion of antigen-primed CD8+ and CD4⁺ T cells in vivo, (2) CD8⁺ T cell compartmentalization, and (3) activation of T cells by antigen presenting dendritic cells⁴⁻⁵.

IL-1 induces signaling by binding to the IL-1 receptor. Upon binding, a shared co-receptor, IL-1RAcP, is recruited by binding to the composite surface of the cytokine and primary receptor complex, resulting in the creation of a ternary complex that initiates a signal transduction cascade.

The IL-1β peptide used in the fusion protein of the present invention need not be the full-length IL-1β protein. However, the IL-1β peptide should include, at a minimum, the IL-1β receptor binding domain, and it should retain the ability to bind to and activate the IL-1 receptor. The ability of an IL-1β peptide to bind to the IL-1 receptor may be assessed using any protein-protein binding assay (including those described above) or using in silico modeling.

IL-1β is produced as an inactive 31 kDa precursor, termed pro-IL-1β. The N-terminal pro-peptide (which is encoded by nucleotides 1-351 of the IL-1β gene) must be cleaved off by the pro-inflammatory protease caspase-1 before mature IL-1β can be secreted. Thus, in preferred embodiments, the IL-1β peptide used in the fusion protein does not include the pro-peptide to facilitate fusokine secretion.

The IL-1β peptide may be derived from an IL-1β protein from any vertebrate animal. Suitable sources of IL-1β proteins include, but are not limited to, humans, non-human primates, cows, cats, dogs, pigs, and rodents. The inventors used a portion of the mouse IL-1β protein that does not include the pro-peptide (SEQ ID NO: 4) in the fusion protein tested in the Examples. Thus, in some embodiments, the IL-1γ peptide has at least 90%, 92%, 94%, 95%, 96%, 98%, or 99% identity to SEQ ID NO:4. In other embodiments, the IL-6 peptide has at least 90%, 92%, 94%, 95%, 96%, 98%, or 99% identity to a portion of the human IL-6 protein (e.g., SEQ ID NO: 12, which does not include the pro-peptide).

Within the IL-6/1 fusokine, the IL-6 peptide is linked to the IL-1β peptide via a linker peptide. As used herein, the term “linker peptide” refers to a peptide that connects two peptide components within a fusion protein. The linker peptide comprises 1 or more amino acid residues, preferably 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or more amino acid residues. The linker peptide may comprise any amino acid sequence that does not substantially hinder the function of the peptide components (i.e., the ability of the IL-6 and IL-1β peptides to bind to their cognate receptors). The inventors used the flexible linker peptide of SEQ ID NO: 6 in the IL-6/1 fusokine tested in the Examples. Thus, in some embodiments, the linker peptide is a flexible linker peptide. In specific embodiments, the linker peptide has at least 90%, 92%, 94%, 95%, 96%, 98%, or 99% identity to SEQ ID NO: 6. As used herein, a “flexible linker peptide” is a linker peptide that has no fixed structure in solution such that the peptide components that it links move freely relative to one another. Examples of preferred amino acid residues for flexible linker peptides include glycine, alanine, serine, threonine, lysine, arginine, glutamine, and glutamic acid. However, the inventors also tested the rigid linker peptide of SEQ ID NO: 17 in the IL-6/1 fusokine, and this linker appeared to work as well as SEQ ID NO: 6 (data not shown). Thus, in other embodiments, the linker peptide is a rigid linker peptide. In specific embodiments, the linker peptide has at least 90%, 92%, 94%, 95%, 96%, 98%, or 99% identity to SEQ ID NO: 17. As used herein, a “rigid linker peptide” is a linker peptide that has a fixed structure in solution.

In some embodiments, the fusion protein comprises from N-terminus to C-terminus: SEQ ID NO: 2 (mouse IL-6), SEQ ID NO: 6 (linker), and SEQ ID NO:4 (mouse IL-1β). In other embodiments, the fusion protein comprises from N-terminus to C-terminus: SEQ ID NO: 10 (human IL-6), SEQ ID NO: 6 (linker), and SEQ ID NO: 12 (human IL-1β). In some embodiments, the fusion protein comprises SEQ ID NO: 8 (mouse IL-6-linker-mouse IL-1γ) or SEQ ID NO: 14 (human IL-6-linker-human IL-1β).

In some embodiments, the fusion protein further comprises a tag. As used herein, a “tag” is a peptide that is genetically grafted onto a fusion protein to serve a particular purpose. Suitable tags for use in the fusion proteins of the present invention include, without limitation, affinity tags for protein purification (e.g., chitin binding protein (CBP), maltose binding protein (MBP), Strep, glutathione-S-transferase (GST), and poly(His) tags), solubilization tags (e.g., thioredoxin (TRX), poly(NANP), MBP, GST), epitope tags for antibody-based detection (e.g., ALFA-tag, V5-tag, Myc-tag, HA-tag, Spot-tag, T7-tag and NE-tag), and fluorescent tags (e.g., green fluorescent protein (GFP), red fluorescent protein (RFP), mKate, TurboRFP) and enzymatic tags (e.g., horseradish peroxidase, alkaline phosphatase, beta-galactosidase, glucose-6-phosphatase, acetylcholinesterase) for visual detection.

Optionally, the tag may be separated from the other components of the fusion protein by a protease cleavage site. In the Examples, the inventors included a TEV protease cleavage site followed by a 6×His tag on the C-terminal end of their fusion protein, which allowed the 6×His tag to be cleaved off using a TEV protease after it was used for protein purification. Those of skill in the art know how to select an appropriate protease cleavage site for such purposes.

### Polynucleotides:

In a second aspect, the present invention provides polynucleotides encoding a fusion protein described herein.

The terms “polynucleotide,” “oligonucleotide,” and “nucleic acid” are used interchangeably to refer a polymer of DNA or RNA. A polynucleotide may be single-stranded or double-stranded and may represent the sense or the antisense strand. A polynucleotide may be synthesized or obtained from a natural source. A polynucleotide may contain natural, non-natural, or altered nucleotides, as well as natural, non-natural, or altered internucleotide linkages. The term polynucleotide encompasses constructs, plasmids, vectors, and the like.

In some embodiments, the polynucleotide encoding the fusion protein is provided as part of a construct in which it is operably linked to a promoter. As used herein, the term “construct” refers a to recombinant polynucleotide, i.e., a polynucleotide that was formed by combining at least two polynucleotide components from different sources, natural or synthetic. For example, a construct may comprise the coding region of one gene operably linked to a promoter that is (1) associated with another gene found within the same genome, (2) from the genome of a different organism, or (3) synthetic. Constructs can be generated using conventional DNA recombination methods.

In some embodiments, the construct is a viral vector. As used herein, a “viral vector” is a recombinant viral nucleic acid that has been engineered to encode a heterologous protein (e.g., a fusion protein described herein). Viral vectors include cis-acting elements that drive the expression of the heterologous protein. Suitable viral vectors are known in the art and include, but are not limited to, adenovirus vectors, adeno-associated virus vectors, pox virus vectors, alpha virus vectors, baculoviral vectors, herpes virus vectors, retrovirus vectors (e.g., lentivirus vectors), Modified Vaccinia virus Ankara vectors, Ross River virus vectors, Sindbis virus vectors, Semliki Forest virus vectors, and Venezuelan equine encephalitis virus vectors. In a preferred embodiment, the viral vector is a lentiviral vector.

As used herein, the term “promoter” refers to a DNA sequence that defines where transcription of a polynucleotide begins. RNA polymerase and the necessary transcription factors bind to the promoter to initiate transcription. Promoters are typically located directly upstream (i.e., at the 5′ end) of the transcription start site. However, a promoter may also be located at the 3′ end, within a coding region, or within an intron of a gene that it regulates. Promoters may be derived in their entirety from a native or heterologous gene, may be composed of elements derived from multiple regulatory sequences found in nature, or may comprise synthetic DNA. A promoter is “operably linked” to a polynucleotide if the promoter is positioned such that it can affect transcription of the polynucleotide. Suitable promoters for use with the present invention include, but are not limited to, constitutive, inducible, temporally regulated, developmentally regulated, chemically regulated, tissue-preferred, and tissue-specific promoters. In some embodiments, the promoter is an elongation factor 1α (EF-1α) promoter, which is known to be one of the strongest promoters for driving expression in mammalian cells. In other embodiments, the promoter is a hybrid CMV enhancer/chicken 3-actin (CBA) promoter, which is commonly used for gene transfer because it provides robust, long-term expression in all cell types. Those of skill in the art understand how to select an appropriate promoter to drive expression of the fusion proteins disclosed herein for a particular application.

In some embodiments, the polynucleotide comprises SEQ ID NO: 1 (i.e., a DNA sequence encoding mouse the IL-6 peptide of SEQ ID NO: 2), SEQ ID NO: 3 (i.e., a DNA sequence encoding the mouse IL-1β peptide of SEQ ID NO: 4), and/or SEQ ID NO: 5 (i.e., a DNA sequence encoding the linker peptide of SEQ ID NO: 6). In other embodiments, the polynucleotide comprises SEQ ID NO: 9 (i.e., a DNA sequence encoding the human IL-6 peptide of SEQ ID NO: 10), SEQ ID NO: 11 (i.e., a DNA sequence encoding the human IL-1β peptide of SEQ ID NO: 12), and/or SEQ ID NO: 5 (i.e., a DNA sequence encoding the linker peptide of SEQ ID NO: 6). In some embodiments, the polynucleotide comprises SEQ ID NO: 7 (i.e., a DNA sequence encoding the mouse IL-6/1 fusokine of SEQ ID NO: 8) or SEQ ID NO: 13 (i.e., a DNA sequence encoding the human IL-6/1 fusokine of SEQ ID NO: 14). In some embodiments, the polynucleotide is the lentiviral vector of SEQ ID NO: 15, which comprises the polynucleotide of SEQ ID NO: 7.

### Cells:

In a third aspect, the present invention provides cells comprising a polynucleotide described herein. Under suitable conditions, the cells express a fusion protein described herein.

A “cell” is the basic unit from which all living things are composed. Every cell comprises cytoplasm (i.e., gelatinous liquid that fills the inside of the cell) enclosed within a membrane. In the cells of the present invention, the polynucleotide may be incorporated into the genome of the cell or may reside in the cytoplasm.

Any cell type may be used with the present invention. In some embodiments, the cell is a host cell used for fusion protein production. In the Examples, the inventors produced IL-6/1 using HEK cells. Thus, in some embodiments, the cell is a HEK cell. In other embodiments, the cell is an immune cell that expresses an IL-6 receptor and/or an IL-1 receptor such that the cell can be activated by the fusion protein after it expresses it. In the Examples, the inventors generated T cells that express the IL-6/1 fusion protein. Thus, in some embodiments, the cells are T cells. In some embodiments, the cell is a cell from a subject into which the polynucleotide was introduced ex vivo.

Any method of protein detection may be used to test whether a cell expresses a fusion protein disclosed herein. Suitable methods for detecting proteins include, without limitation, enzyme-linked immunoassay (ELISA), dot blotting, western blotting, flow cytometry, mass spectrometry, and chromatographic methods.

Methods of Generating a Cell that Expresses the Fusion Protein:

In a fourth aspect, the present invention provides methods of generating a cell that expresses a fusion protein described herein. The methods comprise introducing a polynucleotide that encodes the fusion protein into the cell.

As used herein, “introducing” describes a process by which an exogenous polynucleotide is introduced into a recipient cell. Suitable introduction methods include, without limitation, bacteriophage or viral infection, electroporation, heat shock, lipofection, microinjection, and particle bombardment. In some embodiments, the polynucleotide is a viral vector, and the polynucleotide is introduced into the cell via viral infection.

In some embodiments, the polynucleotide is ultimately inserted into the genome of the cell. In these embodiments, the polynucleotide can be inserted randomly into the genome or targeted to a specific location (e.g., via homologous recombination).

### Methods of Activating a Target Cell:

In a fifth aspect, the present invention provides methods of activating a target cell. The methods comprise contacting the target cell with a fusion protein described herein.

As used herein, the term “activating” refers to a process by which one or more function of a cell is turned on. For example, activated T cells undergo clonal expansion followed by differentiation into effector cells and induction of cell-mediated cytotoxicity and/or cytokine production and release. Detection of any of these functions is evidence that a T cell has been activated.

Any cell that expresses an IL-6 receptor and/or an IL-1 receptor may be activated using these methods. Almost all immune cells express these receptors. Thus, in some embodiments, the target cell is an immune cell. In the Examples, the inventors demonstrate the IL-6/1 fusokine can be used to activate T cells. Thus, in preferred embodiments, the target cell is a T cell.

In the Examples, the inventors demonstrate that IL-6/1 (a) stimulates T cell proliferation (in particular CD8+ T cell proliferation), (b) protects T cells from apoptosis and (c) increases apoptosis or lymphocyte mediated killing of cancer cells. Thus, in some embodiments, the method produces one or more of these outcomes. Cell proliferation may be assessed using any cell proliferation assay and apoptosis may be assessed using any apoptosis assay. Examples of cell proliferation assays include assays in which the number of cells is directly counted over time as well as assays that detect an indicator of proliferation, such as ATP concentration, DNA synthesis, or metabolic activity. Examples of apoptosis assays include caspase activity assays (e.g., assays that detect cleavage of caspase-3 or PARP), phosphatidylserine assays (e.g., Annexin V staining), DNA fragmentation assays (e.g., TUNEL assay), electron microscopy analysis of ultrastructural features, nuclear staining assays (e.g., DAPI or Hoechst staining), and mitochondrial membrane potential assays (e.g., JC-1 staining).

In some embodiments, the method is performed in vitro or ex vivo. For example, in some embodiments, the target cell is activated in cell culture. In other embodiments, the method is performed in vivo in a subject. For example, in some embodiments, the subject has cancer or an infectious disease, and the target cell is activated as a means of treating the cancer or infectious disease.

### Methods of Treating a Disease:

In a sixth aspect, the present invention provides methods of treating a disease in a subject. The methods comprise administering a fusion protein, polynucleotide, construct, or cell described herein to the subject.

As used herein, “treating” describes something that is done to a subject to combat a disease. Treating includes the administration of a composition described herein to prevent the onset of symptoms or complications of the disease, to alleviate the symptoms or complications of the disease, or to eliminate or reduce the effects of the disease. For example, treating cancer in a subject includes reducing, delaying, or preventing cancer growth, reducing tumor volume, and/or reducing, delaying, or preventing metastasis of a tumor. Treating cancer in a subject also includes reducing the number of tumor cells within the subject.

Any disease in which the activation of immune cells will aide in treatment can be treated using the methods of the present invention. Examples of such diseases include infectious diseases and cancers. “Infectious diseases” are diseases that are caused by pathogens such as viruses, bacteria, fungi, and parasites. “Cancers” are diseases that are characterized by an abnormal mass of tissue and in which the growth of the mass surpasses and is not coordinated with the growth of normal tissue. In the case of hematological cancers, this includes a volume of blood or other bodily fluid containing cancerous cells. In the Examples, the inventors demonstrate that IL-6/1 has cytotoxic activity against cancer cells. Thus, in preferred embodiments, the disease is a cancer.

As used herein, the term “administering” refers to the introduction of a substance into a subject's body. Methods of administration are well known in the art and include, but are not limited to, oral administration, transdermal administration, administration by inhalation, nasal administration, topical administration, intravaginal administration, ophthalmic administration, intraaural administration, intracerebral administration, rectal administration, sublingual administration, buccal administration, and parenteral administration, including injectable such as intravenous administration, intra-arterial administration, intramuscular administration, intradermal administration, intrathecal administration, and subcutaneous administration. Administration can be continuous or intermittent.

Polynucleotide-based therapies require that the therapeutic polynucleotide is ferried into cells without eliciting unwanted immune responses. Thus, in some embodiments, a polynucleotide is administered using a drug delivery system. Suitable drug delivery systems include, without limitation, polymer-based, lipid-based, and conjugate-based delivery systems. For example, in some embodiments, the polynucleotide is encapsulated in a polymer- or lipid-based nanoparticle for administration.

The “subject” to which the methods are applied may be a mammal or a non-mammalian animal, such as a bird. Suitable mammals include, but are not limited to, humans, cows, horses, sheep, pigs, goats, rabbits, dogs, cats, bats, mice, and rats. In certain embodiments, the methods may be performed on lab animals (e.g., mice and rats) for research purposes. In other embodiments, the methods are used to treat commercially important farm animals (e.g., cows, horses, pigs, rabbits, goats, sheep, and chickens) or companion animals (e.g., cats and dogs). In a preferred embodiment, the subject is a human. Ideally, the components of the fusion protein are matched to the species of the subject for greater compatibility and reduced risk of antigenicity. For example, the fusion protein ideally comprises a human IL-6 peptide and a human IL-1β peptide if the subject is a human.

The present disclosure is not limited to the specific details of construction, arrangement of components, or method steps set forth herein. The compositions and methods disclosed herein are capable of being made, practiced, used, carried out and/or formed in various ways that will be apparent to one of skill in the art in light of the disclosure that follows. The phraseology and terminology used herein is for the purpose of description only and should not be regarded as limiting to the scope of the claims. Ordinal indicators, such as first, second, and third, as used in the description and the claims to refer to various structures or method steps, are not meant to be construed to indicate any specific structures or steps, or any particular order or configuration to such structures or steps. All methods described herein can be performed in any suitable order unless otherwise indicated herein or otherwise clearly contradicted by context. The use of any and all examples, or exemplary language (e.g., “such as”) provided herein, is intended merely to facilitate the disclosure and does not imply any limitation on the scope of the disclosure unless otherwise claimed. No language in the specification, and no structures shown in the drawings, should be construed as indicating that any non-claimed element is essential to the practice of the disclosed subject matter. The use herein of the terms “including,” “comprising,” or “having,” and variations thereof, is meant to encompass the elements listed thereafter and equivalents thereof, as well as additional elements. Embodiments recited as “including,” “comprising,” or “having” certain elements are also contemplated as “consisting essentially of”′ and “consisting of”′ those certain elements.

Recitation of ranges of values herein are merely intended to serve as a shorthand method of referring individually to each separate value falling within the range, unless otherwise indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure. Use of the word “about” to describe a particular recited amount or range of amounts is meant to indicate that values very near to the recited amount are included in that amount, such as values that could or naturally would be accounted for due to manufacturing tolerances, instrument and human error in forming measurements, and the like. All percentages referring to amounts are by weight unless indicated otherwise.

No admission is made that any reference, including any non-patent or patent document cited in this specification, constitutes prior art. In particular, it will be understood that, unless otherwise stated, reference to any document herein does not constitute an admission that any of these documents forms part of the common general knowledge in the art in the United States or in any other country. Any discussion of the references states what their authors assert, and the applicant reserves the right to challenge the accuracy and pertinence of any of the documents cited herein. All references cited herein are fully incorporated by reference, unless explicitly indicated otherwise. The present disclosure shall control in the event there are any disparities between any definitions and/or description found in the cited references.

The following examples are meant only to be illustrative and are not meant as limitations on the scope of the invention or of the appended claims.

### EXAMPLES

In the following example, the inventors describe the creation of a novel fusion protein that comprises peptides derived from two distinct cytokines, interleukin-6 (IL-6) and interleukin-1 beta (IL-1β). This fusion protein is referred to herein as the “IL-6/1 fusokine”.

IL-6 and IL-1β are both pleiotropic cytokines. IL-6 affects T cell activation, amplification, survival, and polarization¹³. It promotes the expression of T cell attractor chemokines, prevents apoptosis in T cells via STAT3 signaling, and plays a critical role in the accumulation of myeloid derived suppressor cells in tumor microenvironments. IL-1β promotes (1) expansion of antigen-primed CD8^{m} and CD4^{m} T cells in vivo, (2) CD8⁺ T cell compartmentalization, and (3) activation of T cells by antigen presenting dendritic cells⁴⁻⁵. Thus, the IL-6/1 fusokine harnesses all these immune-stimulatory mechanisms to promote T cell activation, survival, and tumoricidal activities.

### Generation of IL-6/1

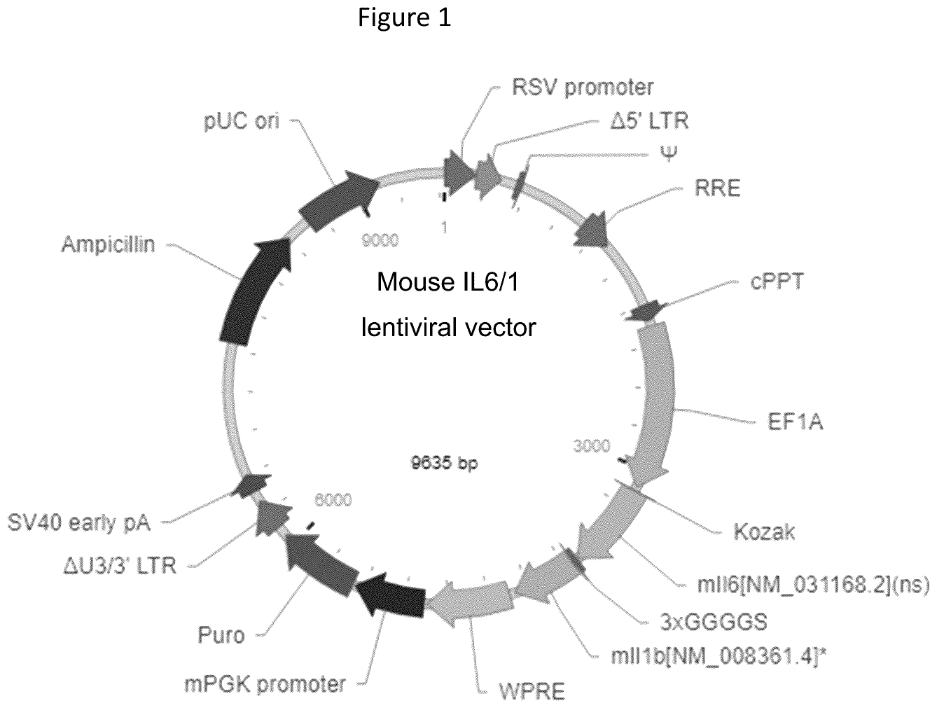

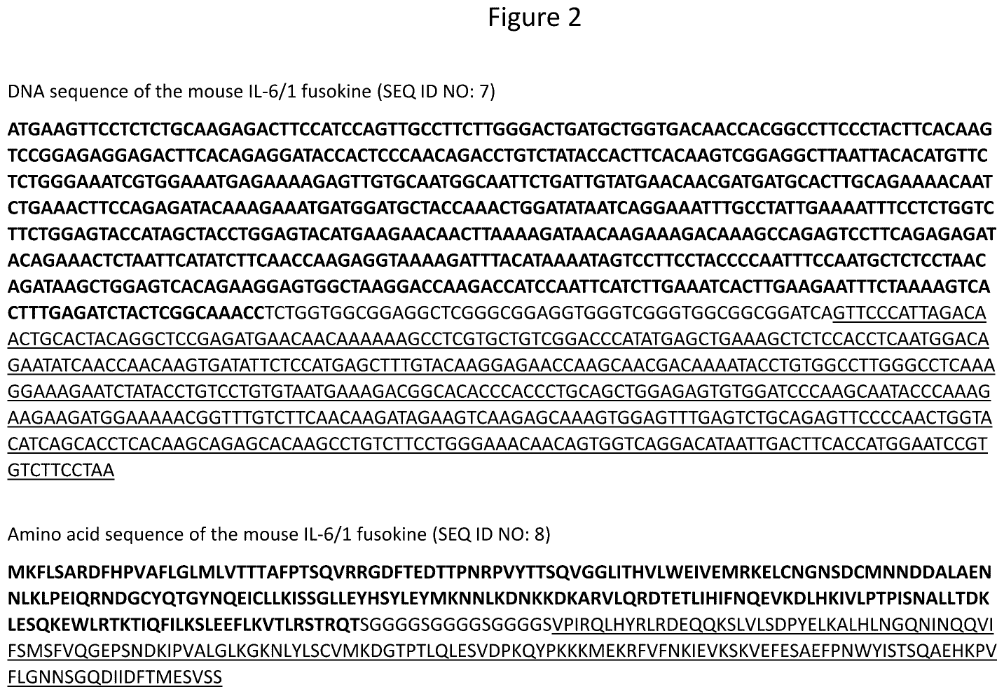

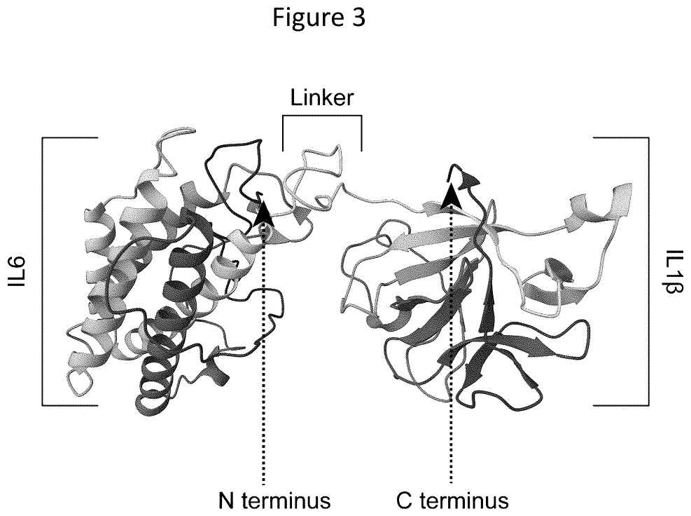

The IL-6/1 fusokine tested in this example comprises from N-terminus to C-terminus: the full-length mouse IL-6 protein (SEQ ID NO: 2), a 16-amino acid flexible peptide linker (SEQ ID NO: 6), and a portion of the mouse IL-1β protein (SEQ ID NO: 4, which does not include the pro-peptide of IL-1β to facilitate fusokine secretion). In some instances, a TEV protease cleavage site followed by a 6×-HIS tag were included at the C-terminus of IL-1β to allow for purification of the fusion protein. The cDNA sequence encoding IL-6/1 was cloned in frame into the lentiviral expression vector pLV from which the transgene is expressed as a single open reading frame regulated by the full-length EF1A promoter. A vector map is provided in FIG. 1 and a detailed description of the vector components is provided in Table 1 below. Additionally, the sequence of IL-6/1 is presented in FIG. 2, and the predicted secondary structure of IL-6/1 is shown in FIG. 3.

TABLE 1
Lentiviral vector components

| Name | Position | Size (bp) | Description | Function/Notes |
|---|---|---|---|---|
| RSV | 1-229 | 229 | Rous sarcoma | Strong promoter; drives |
| promoter | | | virus enhancer/ | transcription of viral |
| | | | promoter | RNA in packaging cells. |
| Δ5′ | 230-410 | 181 | Truncated HIV-1 | Allows for transcription |
| LTR | | | 5′ long terminal | of viral RNA and its |
| | | | repeat | packaging into virus. |
| Ψ | 521-565 | 45 | HIV-1 packaging | Allows for packaging of |
| | | | signal | viral RNA into virus. |
| RRE | 1075-1308 | 234 | HIV-1 Rev | Rev protein binding site |
| | | | response | that allows for Rey- |
| | | | element | dependent nuclear export |
| | | | | of viral RNA during |
| | | | | viral packaging. |
| cPPT | 1803-1920 | 118 | Central | Facilitates nuclear |
| | | | polypurine | import of cDNA through |
| | | | tract | a central DNA flap. |
| EF1A | 1959-3137 | 1179 | Human eukaryotic | Strong promoter, allows |
| | | | translation | for constitutive |
| | | | elongation factor | expression of transgene |
| | | | 1 α1 promoter | in various cell types, |
| | | | | including primary cells. |
| Kozak | 3162-3167 | 6 | Kozak translation | Facilitates translation |
| | | | initiation | initiation of ATG start |
| | | | sequence | codon downstream of the |
| | | | | Kozak sequence. |
| mIl6[NM_031168.2](ns) | 3168-3800 | 633 | Full-length mouse | Stop codon at the C- |
| | | | IL-6 cDNA | terminus was removed to |
| | | | | allow continuous |
| | | | | transcription of the |
| | | | | following fusokine |
| | | | | components. |
| 3xGGGGS | 3801-3848 | 48 | Linker comprising | Commonly used |
| | | | 3 tandem GGGGS | flexible linker. |
| | | | (SEQ ID NO: 16) | |
| | | | sequences | |
| mIl1b[NM_008361.4]* | 3849-4307 | 459 | Mouse IL-1β | The pro-peptide |
| | | | cDNA | encoding sequence (i.e., |
| | | | | nucleotides 1-351) was |
| | | | | removed; mature form of |
| | | | | IL-1β. |
| WPRE | 4346-4943 | 598 | Woodchuck | Enhances lentivirus |
| | | | hepatitis virus | stability in packaging |
| | | | posttranscriptional | cells, leading to higher |
| | | | regulatory element | titer of packaged virus; |
| | | | | increases expression of |
| | | | | transgenes. |
| mPGK | 4962-5472 | 511 | Mouse | Medium-strength |
| promoter | | | phosphoglycerate | promoter; used to |
| | | | kinase 1 promoter | express selection marker |
| | | | | in the transduced cells. |
| Puro | 5485-6084 | 600 | Puromycin | Makes cells resistant to |
| | | | resistance gene | puromycin. |
| ΔU3/3′ | 6155-6389 | 235 | Truncated HIV-1 | Allows for packaging of |
| LTR | | | 3′ long terminal | viral RNA into virus; |
| | | | repeat | self-inactivates the 5′ |
| | | | | LTR via a copying |
| | | | | mechanism during viral |
| | | | | genome integration; |
| | | | | contains polyadenylation |
| | | | | signal for transcription |
| | | | | termination. |
| SV40 | 6462-6596 | 135 | Simian virus | Allows for transcription |
| early pA | | | 40 early | termination and |
| | | | polyadenylation | polyadenylation of |
| | | | signal | mRNA transcribed by |
| | | | | Pol II RNA polymerase. |
| Ampicillin | 7550-8410 | 861 | Ampicillin | Makes cells resistant to |
| | | | resistance gene | ampicillin. |
| pUC ori | 8581-9169 | 589 | pUC origin of | Facilitates plasmid |
| | | | replication | replication in E. coli; |
| | | | | regulates high-copy |
| | | | | plasmid number |
| | | | | (500-700). |

To determine the effects of the spatial orientation of the individual cytokines (i.e., IL-6 and IL-1β) within the fusion protein, a second lentiviral vector was generated wherein the relative positions of the peptides was reversed, such that the IL-1β peptide was positioned N-terminal to the IL-6 peptide. This reversed fusion protein is referred to herein as the IL-1/6 fusokine. It comprises the same linker peptide as the IL-6/1 fusokine.

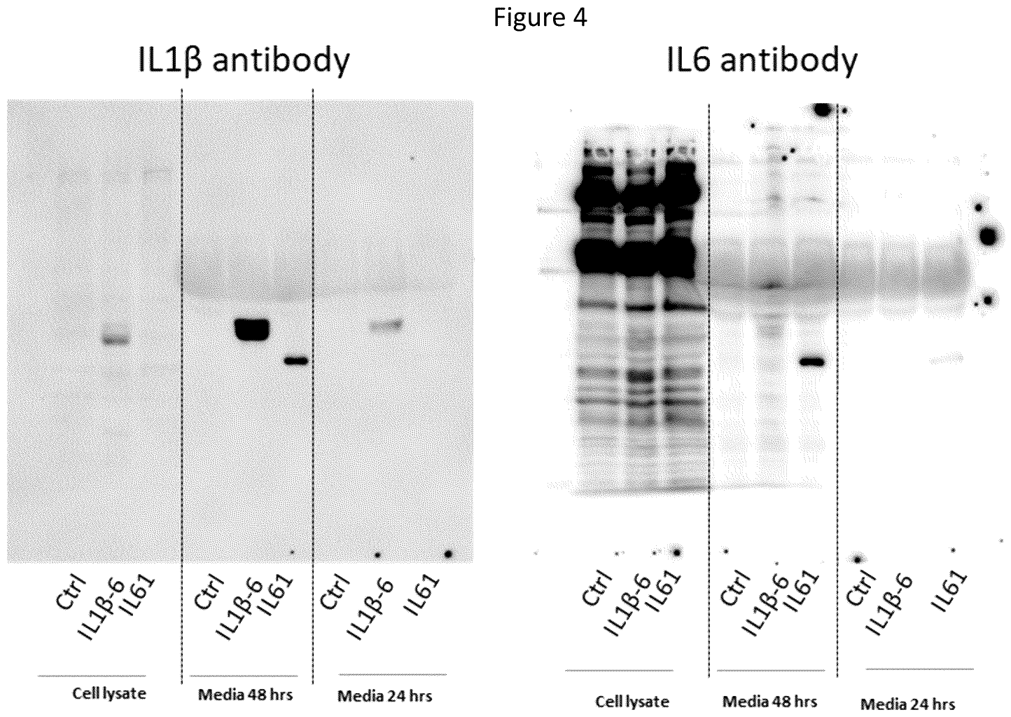

To test for fusokine expression, HEK cells were transduced with lentiviral vector encoding a fusokine (i.e., IL-6/1 or IL-1/6). Media collected from the cell culture after 24 or 48 hours of culturing and lysate collected after 48 hours of culturing were analyzed via western blot using antibodies against IL-1β and IL-6. While the anti-IL-6 antibody failed to recognize its target antigen within the IL-1/6 fusokine, the anti-IL-1β antibody could detect its target antigen within both fusokines (FIG. 4). This indicates that the epitope structure of IL-6 is retained in IL-6/1 but not in IL-1/6. Accordingly, the IL-6/1 fusokine was selected for further study. The results of this western blot further demonstrate that IL-6/1 is readily secreted from HEK cells transduced with the lentiviral vector.

To generate fusokine protein for use in further experiments, HEK cells were transduced with lentiviral vector encoding the IL-6/1 fusokine or a control protein (GFP). Positively transduced cells were selected via antibiotic selection. High fusokine expressing clones were identified via clonal selection followed by IL-6 ELISA and were expanded and cultured for 48 hours in fresh culture media for protein production. The culture media was collected, briefly centrifuged to remove cellular debris, and concentrated 100-fold using a 30 KDa cutoff column. Fusokine concentration was then determined by ELISA, and the concentrate was aliquoted and stored at −80° C. For cell-based assays, the protein concentrate was initially thawed on ice and then at room temperature prior to adding to recipient cells.

### Characterization of IL-6/1

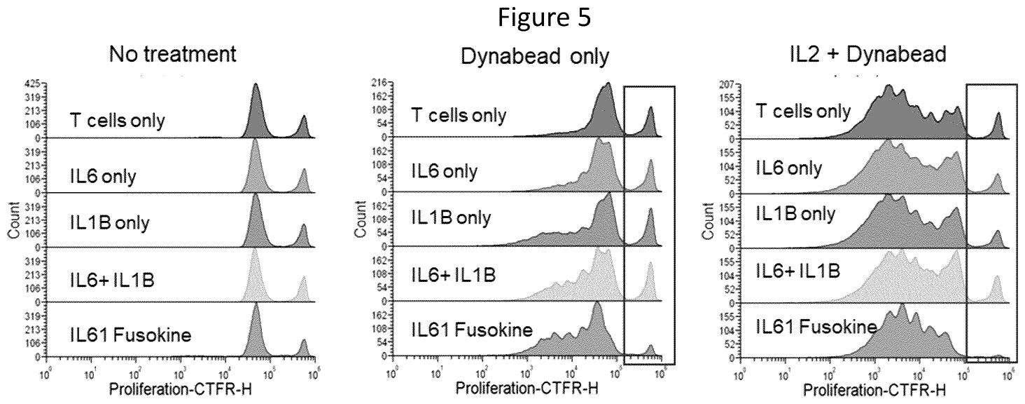

The ability of the IL-6/1 fusokine to activate T cells was assessed. Human peripheral blood mononuclear cells (PBMCs) were collected from a healthy donor and CD3⁺ T cells were purified. The cells were labeled with CellTrace™ Far Red (CTFR) dye to track proliferation. In a first experiment, these cells were activated via treatment with CD3-CD28 Dynabeads® (magnetic beads), alone or in combination with IL-2 cytokine, in the presence of IL-6, IL-1β, a combination of IL-6 and IL-1β, or the IL-6/1 fusokine, and cell proliferation was analyzed using flow cytometry. The results of this experiment show that IL-6/1 treatment stimulated a subset of the PBMC T cells to proliferate (FIG. 5).

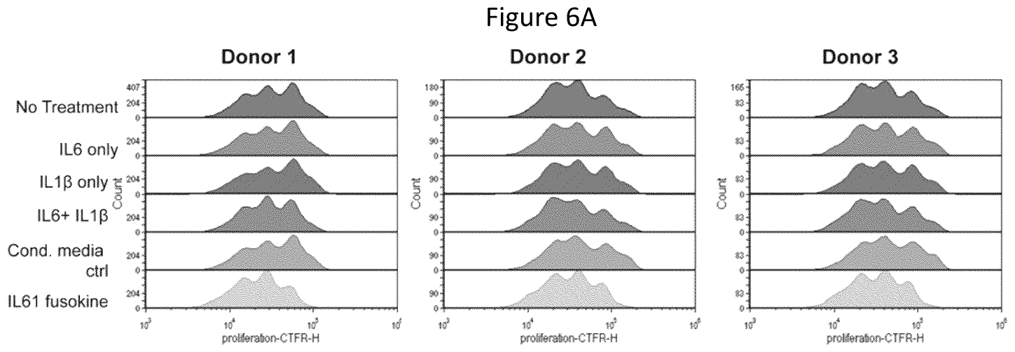

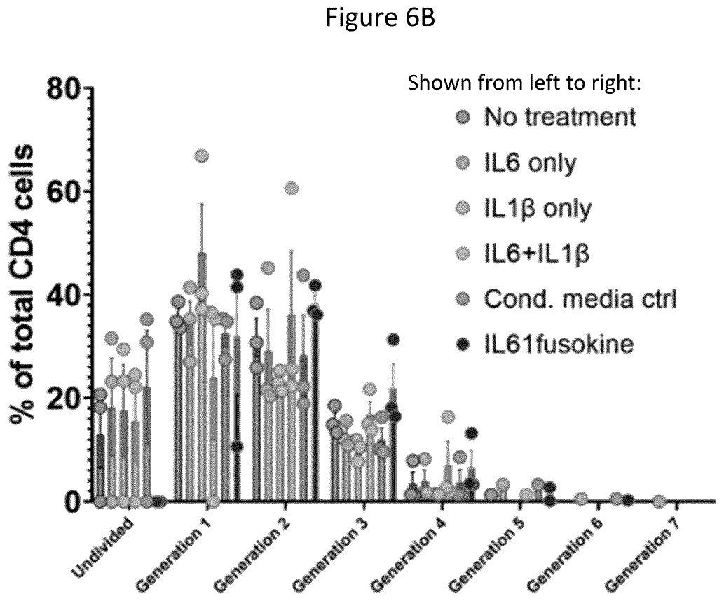

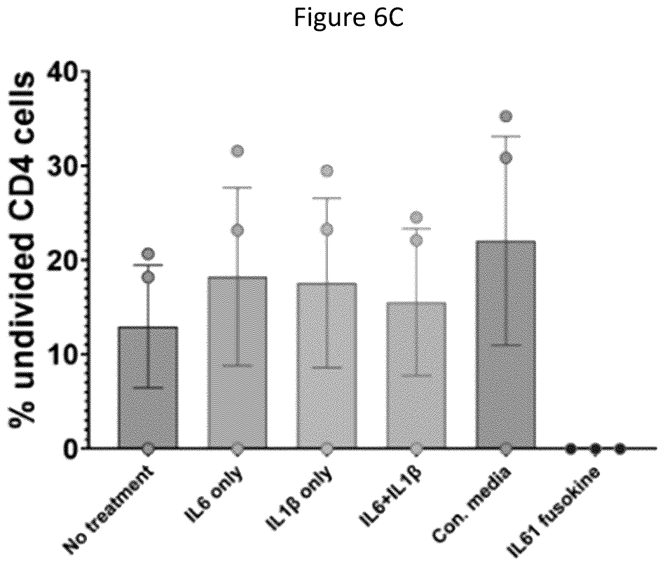

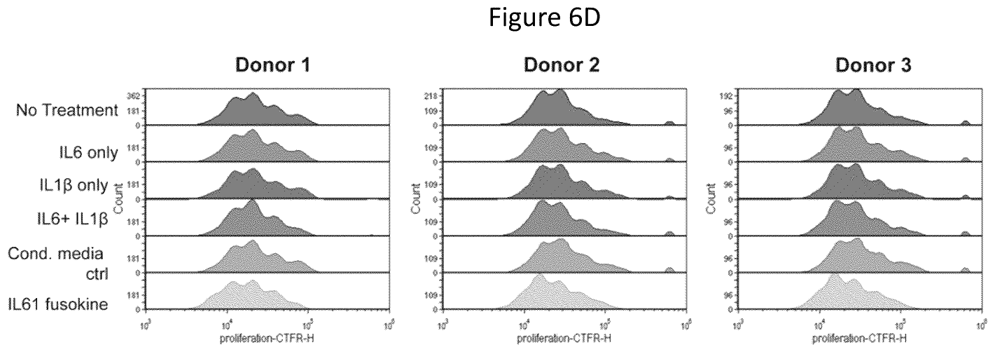

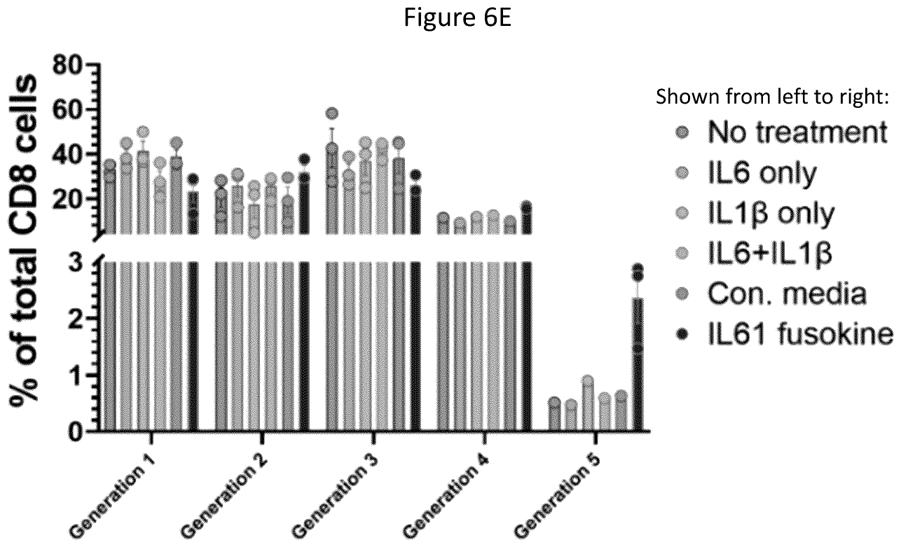

The effects of the IL-6/1 fusokine on T cell proliferation were further characterized using blood-derived T cells from multiple human donors. In this experiment, the cells were labeled with CTFR dye to track proliferation and were activated via treatment with an anti-CD3/anti-CD28 antibody cocktail in combination with IL-2 cytokine in the presence of IL-6, IL-1β, a combination of IL-6 and IL-1β, or the IL-6/1 fusokine. The proliferation of different subtypes of T cells (i.e., CD4⁺ and CD8⁺) was analyzed using flow cytometry after 72 hours of culturing. The IL-6/1 fusokine had different effects on the CD4⁺ and CD8⁺ cell subsets. For the CD4⁺ cells, IL-6/1 caused cells from all donors to proliferate and there were few undivided cells, whereas the other treatments produced a similar effect in only one of the three donors (FIGS. 6A-6C). In contrast, all tested treatments resulted in proliferation of CD8⁺ cells with no undivided cells (FIG. 6D). However, treatment with IL-6/1 produced the highest percentage of proliferating cells in the last tested generation (FIG. 6E). These results demonstrate that the IL-6/1 fusokine enhances activation-mediated proliferation of CD4⁺ and CD8⁺ T cells in a different manner.

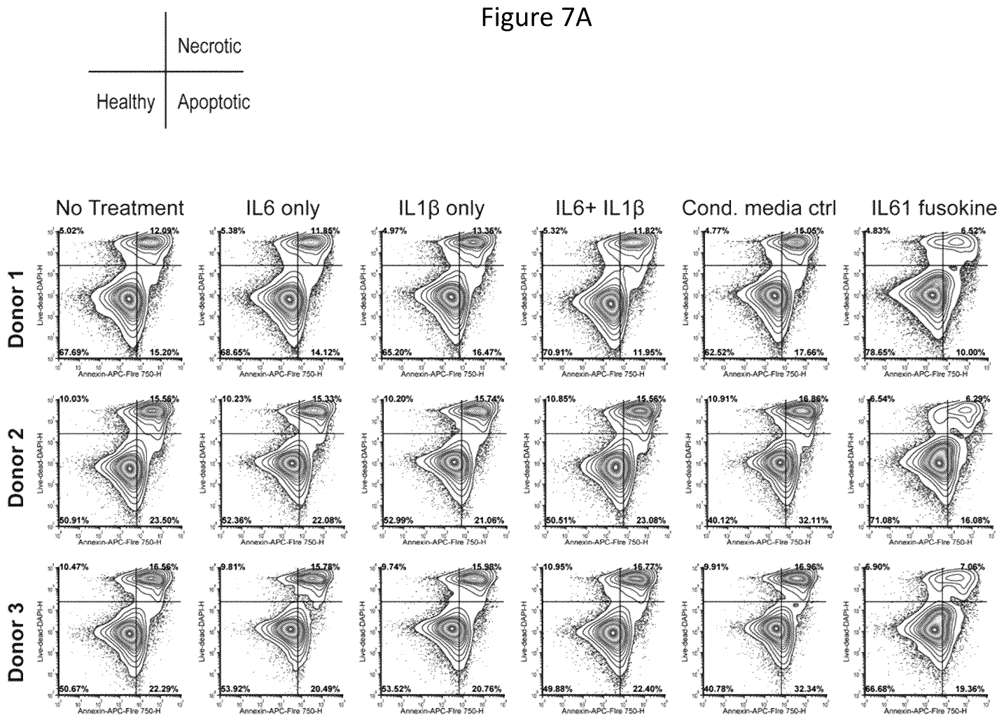

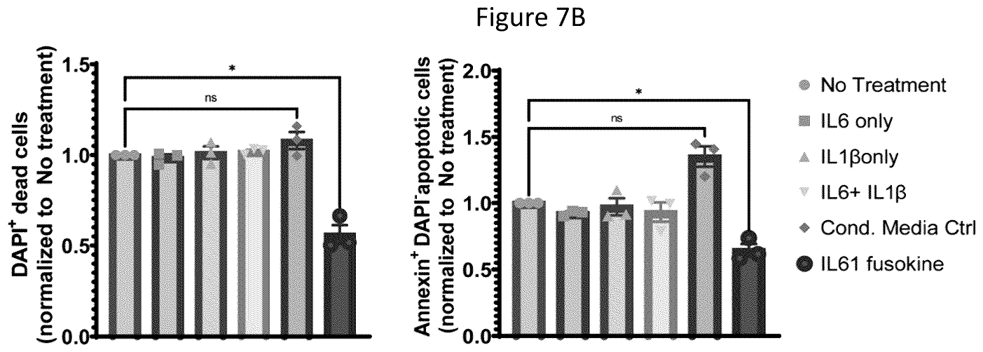

To assess the effects of the IL-6/1 fusokine on T cell apoptosis, the CD3⁺ T cells were activated via treatment with IL-6, IL-1β, a combination of IL-6 and IL-1β, or the IL-6/1 fusokine. After 96 hours of culturing in the presence of an anti-CD3/anti-CD28 antibody cocktail, cells were stained with annexin and DAPI to measure apoptosis using flow cytometry. The results of this experiment show that, of the tested treatments, IL-6/1 treatment results in the lowest amount of annexin-positive apoptotic cells and DAPI-positive necrotic cells (FIGS. 7A-7B), indicating that IL-6/1 protects T cells from activation-induced apoptosis.

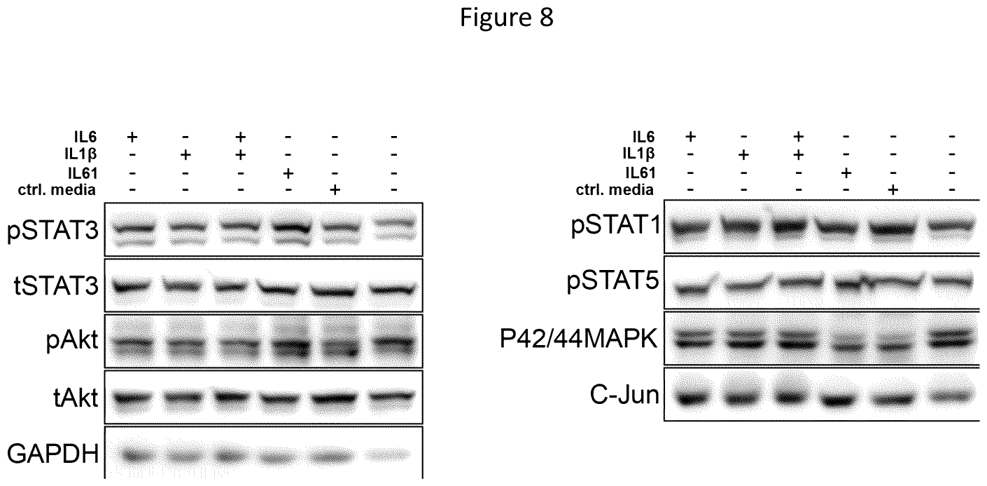

To assess the effects of the IL-6/1 fusokine on the JAK-STAT signaling pathway in T cells, human PBMCs were collected from a healthy donor and CD3⁺ T cells were purified. Cells were induced to proliferate using anti-CD3 and anti-CD28 antibodies in the presence of IL-6, IL-1β, a combination of IL-6 and IL-1β, or the IL-6/1 fusokine. Conditioned media obtained from mock vector-transduced HEK cells was used as a control for the effects of proteins secreted by wild-type HEK cells. After 72 hours of culturing, cells were collected, lysed, and subjected to western blot to analyze levels of phosphorylated STAT3 (pSTAT3) and phosphorylated AKT (pAKT). The results of this experiment show that pSTAT3 and pAKT levels are higher following treatment with the IL-6/1 fusokine as compared to the other tested treatments (FIG. 8). This indicates that the IL-6/1 fusokine strongly activates the STAT3-AKT signaling pathway in activated human PBMC T cells and elucidates a mechanism by which IL-6/1 alters the function of PBMCs.

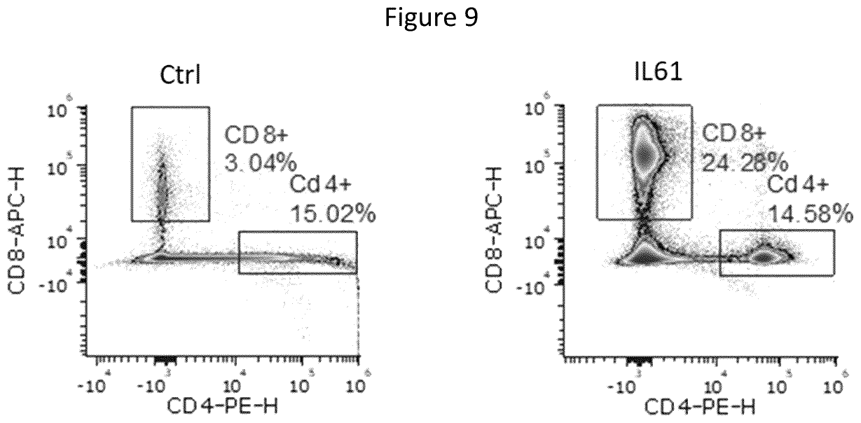

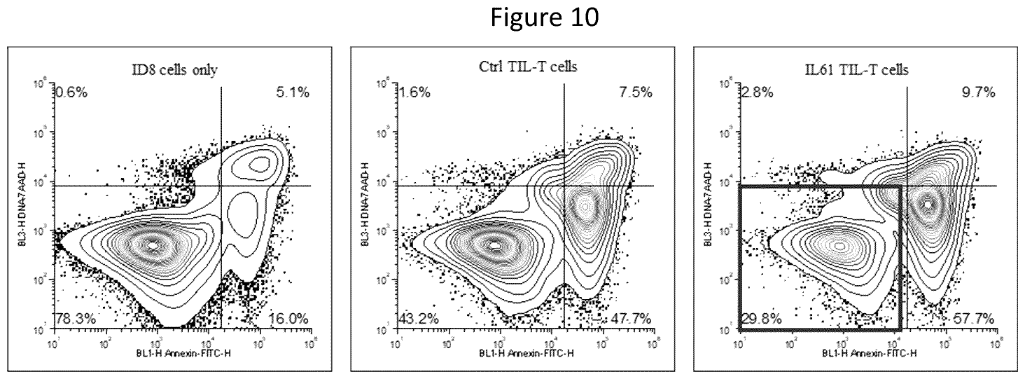

Finally, the anti-cancer activity of the IL-6/1 fusokine was assessed. A mouse model of ovarian cancer was generated by injecting ID8 carcinoma cells into mice. Tumor infiltrating lymphocyte (TTL)-containing ascetic fluid was collected from the mice about 60 days after injection. TTL-T cells were purified and transduced with control lentivirus or lentivirus encoding the IL-6/1 fusokine. Flow cytometry analysis reveals that the IL-6/1 fusokine induces murine CD8⁺ TIL proliferation (FIG. 9). The transduced TIL-T cells were co-cultured with ID8 cells in vitro, and the cells were stained for annexin and 7AAD to detect apoptosis. While the control TIL-T cells showed some cytotoxic effects, the IL-6/1-transduced TIL-T cells caused a further reduction in the viable ID8 cell population (FIG. 10), demonstrating that IL-6/1-transduced TTL-T cells have strong in vitro cytotoxic activity against murine ovarian cancer cells.

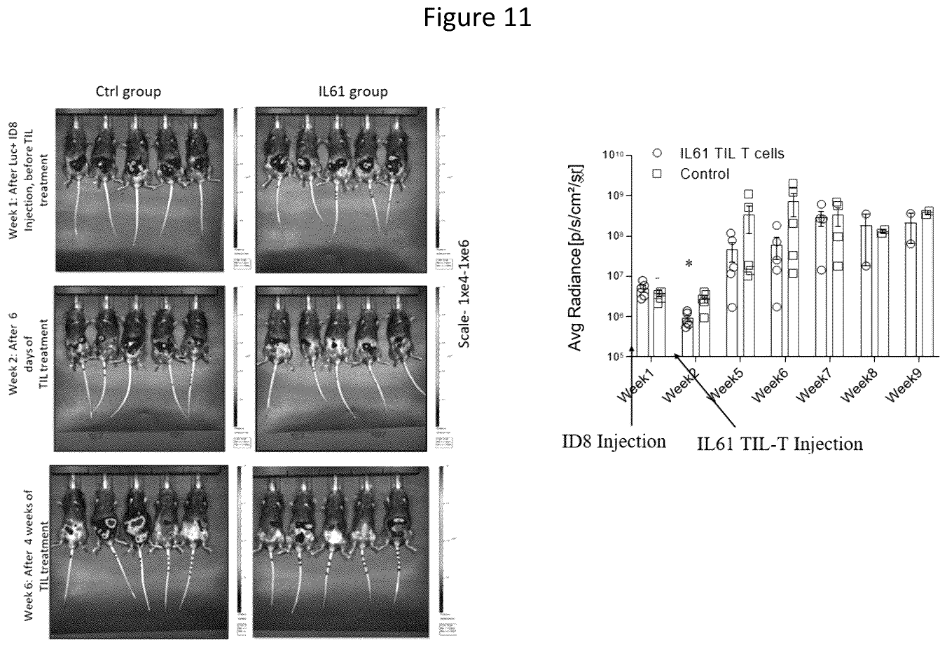

To test the in vivo cytotoxic activity of the IL-6/1-transduced TTL-T cells, another batch of mice was injected with 1×10⁶ luciferase-expressing ID8 cells. After 4 days, these mice were injected with PBS or with IL-6/1-transduced TIL-T cells. In vivo imaging revealed that IL-6/1-transduced TTL-T cells have tumoricidal activity against the murine ovarian cancer cells (FIG. 11).

### REFERENCES

1. Tanaka, T., Narazaki, M. & Kishimoto, T. IL-6 in inflammation, immunity, and disease. Cold Spring HarbPerspect Biol 6, a016295, doi:10.1101/cshperspect.a016295 (2014).
2. Jones, S. A. & Jenkins, B. J. Recent insights into targeting the IL-6 cytokine family in inflammatory dis-eases and cancer. Nat Rev Immunol 18, 773-789, doi:10.1038/s41577-018-0066-7 (2018).
3. Hirano, T. IL-6 in inflammation, autoimmunity and cancer. Int Immunol 33, 127-148, doi:10.1093/intimm/dxaa078 (2021).
4. Bent, R., Moll, L., Grabbe, S. & Bros, M. Interleukin-1 Beta-A Friend or Foe in Malignancies? Int J Mol Sci 19, doi:10.3390/ijms19082155 (2018).
5. Rebe, C. & Ghiringhelli, F. Interleukin-1beta and Cancer. Cancers (Basel) 12, doi:10.3390/cancers12071791 (2020).

What is claimed:

1. A fusion protein comprising from N-terminus to C-terminus:
a) an interleukin-6 (IL-6) peptide;
b) a linker peptide; and
c) an interleukin-1 beta (IL-1β) peptide.

2. The fusion protein of claim 1, wherein the IL-6 peptide has at least 95% identity to SEQ ID NO: 2 or SEQ ID NO: 10.

3. The fusion protein of claim 1, wherein the IL-1β peptide has at least 95% identity to SEQ ID NO: 4 or SEQ ID NO: 12.

4. The fusion protein of claim 1, wherein the linker peptide has at least 95% identity to SEQ ID NO: 6.

5. The fusion protein of claim 1, wherein the fusion protein comprises SEQ ID NO: 8 or SEQ ID NO: 14.

6. A polynucleotide encoding the fusion protein of claim 1.

7. A construct comprising the polynucleotide of claim 6 operably linked to a promoter.

8. The construct of claim 7, wherein the promoter is an elongation factor 1 α (EF-1α) promoter.

9. The construct of claim 7, wherein the construct is a viral vector.

10. The construct of claim 9, wherein the viral vector is a lentiviral vector

11. A cell comprising the polynucleotide of claim 6.

12. The cell of claim 11, wherein the cell is a T cell.

13. A method of generating a cell that expresses the fusion protein of claim 1, the method comprising introducing a polynucleotide encoding the fusion protein into the cell.

14. A method of activating a target cell, the method comprising contacting the target cell with the fusion protein of claim 1.

15. The method of claim 14, wherein the target cell expresses an IL-6 receptor and/or an IL-10 receptor.

16. The method of claim 15, wherein the target cell is a T cell.

17. The method of claim 16, wherein the method:
a) stimulates T cell proliferation; and/or
b) protects T cells from apoptosis.

18. The method of claim 14, wherein the method is performed in vitro or ex vivo.

19. The method of claim 14, wherein the method is performed in vivo in a subject.

20. The method of claim 19, wherein the subject has cancer or an infectious disease.

21. A method of treating a disease in a subject, the method comprising administering the fusion protein of claim 1 to the subject.

22. The method of claim 21, wherein the disease is cancer or an infectious disease.
